(19) United States
(12) Patent Application Publication
(10) Pub. No.: US 2008/0275359 A1
(43) Pub. Date: Nov. 6, 2008

(54) Mobile in vivo brain scan and analysis system

(76) Inventors: Frederick W. Mintz, Chatsworth, CA (US); Philip I. Moynihan, La Canada, CA (US)

Correspondence Address: TOPE-MCKAY & ASSOCIATES, 23852 PACIFIC COAST HIGHWAY #311, MALIBU, CA, 90265, US

(21) Appl. No.: 12/217,463

(22) Filed: Jul. 3, 2008

Related U.S. Application Data
(63) Continuation of application No. 11/726,403, filed on Mar. 20, 2007.
(60) Provisional application No. 60/783,938, filed on Mar. 20, 2006.

(51) Int. Cl. A61B 5/0476

(52) U.S. Cl. 600/544

(57) ABSTRACT

Described is a mobile in vivo brain scan and analysis system. The system includes a data collection subsystem and a data analysis subsystem. The data collection subsystem is formed to collect brain data that is reflective of firing neurons in a mobile subject and transmit the brain data to the data analysis subsystem. The data analysis subsystem is configured to generate and display a three-dimensional image that depicts a location the firing neurons. The data analysis subsystem is also configured to compare the brain data against a library of brain data to detect an anomaly in the brain data, and notify a user of any detected anomaly in the brain data.

### PRIORITY CLAIM

The present application is a Continuation-in-Part patent application, claiming the benefit of priority of U.S. patent application Ser. No. 11/726,403, filed on Mar. 20, 2007, entitled, “Mobile Electroencephalograph Data Collection and Diagnosis System,” which is a non-provisional patent application, claiming the benefit of priority to U.S. Provisional Application No. 60/783,938, filed on Mar. 20, 2006, entitled, “Mobile in vivo EEG data collection and diagnoses comparison system.”

### STATEMENT OF GOVERNMENT INTEREST

The invention described herein was made in the performance of work under a NASA contract, and is subject to the provisions of Public Law 96-517 (35 USC 202) in which the Contractor has elected to retain title.

### BACKGROUND OF THE INVENTION

(1) Field of Invention

The present invention relates to a electroencephalographic (EEG) data analysis system and, more specifically, to a mobile in vivo brain scan system that is configured to collect remote and mobile EEG data for real-time analysis.

(2) Description of Related Art

Historically, human-brain dysfunctions have been diagnosed by psychiatric and psychological professionals in terms of behavioral characteristics. This approach to diagnoses catalogs the incidences of observed behavior in statistical correspondence to those listed in the Diagnostic and Statistical Manual of Mental Disorders, Fourth Edition (DSM). Better known as the DSM-IV-TR, the manual is published by the American Psychiatric Association and covers all mental health disorders for both children and adults. It also lists known causes of these disorders, statistics in terms of gender, age at onset, and prognosis as well as some research concerning optimal treatment approaches. The Manual is re-published about every five years. It establishes standard definitions of pathologic behavior but, over the years, often substantially changes the definitions of abnormal vs. normal behavior.

Therapies carried out by professional clinicians range from permanent institutionalization through in-patient hospital treatment to various, out-patient behavior modification techniques. Often, psychiatric physicians prescribe chemical drug therapies in order to modify or manage symptomatic behavior. These drugs are meant to alter neuro-chemical activity in the brain. Validation of the efficacy of these drug treatments is, however, still observational. Although these clinicians may carry out a protocol of pre- and post-therapy blood tests to ascertain metabolic balances juxtaposed against behavioral changes, the diagnoses are still observational and subjective. This approach to diagnosis defines the “Subjective versus Objective Problem.” What is needed is a more scientific approach to diagnosis than observation-based determinations.

It is generally recognized that upwards of 80 percent of all neuro-scientists have existed only in the past 30 years. It has only been within the past 15 years that sophisticated brain imaging techniques have been invented and used in attempts to inform researchers and clinicians about brain activity versus behavior. The discovery of “brain waves” is attributed to Richard Canton, an English researcher in the year 1875. Electro-Encephalography (EEG) as a technique for detecting brain activity in humans and is attributed to Hans Berger in the year 1929. Modern brain imaging techniques date from the mid-1970's, with the advent of Magnetic Resonance imaging (MRI). The current state-of-the-art for brain imaging is called function Magnetic Resonance Imaging (fMRI).

Imaging (fMRI) combines the sum of constructs of evoked potential events (at the bundled synapses level) with the static images of an MRI image. However, fMRI is not a mobile, physical activity related, real-time diagnostic tool. In other words, while fMRI provides data related to sight, sound, and some thought, it confines the user to a large machine and does not provide for brain analysis while moving. Thus, current fMRI techniques do not provide for an analysis during the range of human behaviors, particularly for children and adults with serious behavioral or mental problems.

The overwhelming complexity of the mammalian brain, juxtaposed against the current (noninvasive) state-of-the-art in brain imaging, associated with scientifically characterizing various brain activities, still defines the “Objective versus Subjective Problem.” Thus, what occurs in the brain from a physiological and chemical point of view, relating to active physical and psychological behavior, is still largely unknown. The reason for most of the imprecise diagnoses is due to “The Subjective versus Objective Problem.”

The substantial overlap of observed behaviors, characterized in the List of Clinical Syndromes, Developmental, and Personality Disorders in the DSM, is legion. It is estimated by some researchers that more than 25 percent of the World's population suffers from mental/emotional problems. It is also estimated that 47 percent of those requiring interventional mental care do not receive it. More than 28 percent of the Member Nations of the World Health Organization do not have budget items for mental health. Even in countries like the United States of America, the statistics seem ineluctable. Most of the mentally disabled do not receive effective intervention therapies due to well-recognized and closely related problems, such as poverty, lack of medical insurance coverage, and inaccurate diagnoses.

Currently, scientific studies relating to the brain take place primarily in university departments of medicine, biology, psychology, engineering, information science, philosophy, and (interestingly) music. The wide range of these academic research interests is beginning to generate a growing amount of much needed, interdisciplinary research and data sharing.

In addition to the need to more precisely define the standardized diagnoses cataloged in the DSM, there is a need to study the neuronal activity of the brain in real-time as it responds to a wide range of internal and external stimuli (as described further below). There is a need, for instance, to study the impact of psychological and physical trauma on the developing brain. Additionally, there is a need for brain studies of high-risk job candidates, such as astronauts to qualify them for space flight as well as astronauts working in outer space. Further, there is an urgent need to study the possible causes and results of a broad range of Autistic Spectrum Disorders in adults and children.

The central and peripheral nervous systems are marvelous detector systems for sensing minuscule changes in the external environment in which the human body exists. In many respects, the human auditory, visual, and olfactory systems are far more sensitive than our most sensitive detector instruments. Indeed, the human eye can detect changes of a single photon! The olfactory bulbs and auditory processing systems in the brain are similarly sensitive. However, the integration and processing times, necessary to become consciously aware of these sensory inputs, is often dangerously long. This is because our own, “experiential training,” inhibits the conscious compilation of these data in the prefrontal cortex, which is where they are first evaluated against memorized experiences in frontal cortex and then sent back to other parts of the brain to “take action.”

This is why external stimuli (such as potentially dangerous sights, sounds, smells, movements, skin pressure) and all of the other external somatosensory inputs, as well as dysfunctional internal sensory stimuli (such as an irregular heartbeat, breathing, blood pressure, planar orientation, gravity etc., carried by the proprioceptive nervous system) are largely “ignored,” until it is consciously decided that they have become too critical. Humans cannot detect (at the conscious level) most of these “early warning signals.” Humans have learned, over millions of years, to ignore many of these minute stimuli in favor of adapting to or modifying the surrounding environment. This may explain why animals are far more sensitive to external stimuli; because they have not learned to ignore or modify environmental change like humans (e.g., the recent tragic tsunami events in the Far East, where all of the indigenous animals ran inland long before the first tsunami hit the beaches).

Thus, when things begin to “go wrong” on Earth or in Outer Space, they often start as a small anomaly; like the distant, imperceptible roar of sounds in the sea (a precursor to a tsunami), or a low frequency, slowly moving ground wave (a precursor of an earthquake), or the small reduction in pressure on the human skin (like the beginning of a crack/leak in a space suit). All of these small anomalies induce subtle neuronal changes, which the Central Nervous System (CNS) will detect and record long before the Earth bound or spacecraft instruments or commercial environmental safety systems, calibrated to “acceptable ranges,” detect such dangerous stimuli.

Therefore, in addition to the reasons listed above for detecting and monitoring psychological conditions, there is an urgent need to detect and accurately identify, in real-time, brain related physiological anomalies and disabilities.

### SUMMARY OF INVENTION

The present invention is a mobile in vivo brain scan and analysis system. The system comprises a data analysis subsystem having one or more processors that are configured to receive brain data from a remote data collection subsystem. The brain data is reflective of firing neurons in a mobile subject. The data analysis subsystem is further configured to generate and display a three-dimensional image that depicts a location of the firing neurons.

In another aspect, the data analysis subsystem is further configured to compare the brain data against a library of brain data to detect an anomaly in the brain data and notify a user of any detected anomaly in the brain data. The anomaly is indicative of an abnormal brain function.

In yet another aspect, the present invention further comprises a data collection subsystem that is formed to collect brain data and transmit the brain data to the data analysis subsystem. The data collection subsystem is further formed to transmit the brain data wirelessly to the data analysis subsystem.

Additionally, the data collection subsystem further comprises a helmet with an array of electrodes, with each of the electrodes being formed to detect electroencephalograph (EEG) data from a user.

In another aspect, the data analysis subsystem further comprises a receiver system and a data processing system. The receiver system is configured to receive the transmitted brain data from the data collection subsystem. The data processing system has a relational database management system (RDBMS) controller for connecting with and operating an RDBMS having a library of brain data. The data processing system is also configured to receive the brain data from the receiver system and compare the brain data to the RDBMS to detect an anomaly in the brain data.

In another aspect, the data analysis subsystem is further configured to compare a detected anomaly in the brain data with an RDBMS to generate a diagnosis of the detected anomaly. The data analysis subsystem is further configured to compare the three-dimensional image with a RDBMS having a library of three-dimensional images to detect an anomaly in the brain data.

As can be appreciated by one skilled in the art, the present invention also comprises a method for forming and using the system described herein. For example, the present invention includes a method for detecting anomalous brain activity. The method comprises a plurality of acts of performing the operations described below.

### BRIEF DESCRIPTION OF THE DRAWINGS

The objects, features and advantages of the present invention will be apparent from the following detailed descriptions of the various aspects of the invention in conjunction with reference to the following drawings, where:

FIG. 1 is a cross-sectional view of an electrode according to the present invention;

FIG. 2 is a cross-sectional, rear-view of a helmet, illustrating a shock absorbent lining of the helmet according to the present invention;

FIG. 3 is a right, side-view of the shock absorbent lining of the helmet according to the present invention;

FIG. 4 is a cross-sectional, left side-view of the shock absorbent lining of the helmet according to the present invention;

FIG. 5 is an exploded-view of components of an EEG system according to the present invention; and

FIG. 6 is a data flow diagram of a mobile in vivo EEG brain scan system according to the present invention.

### DETAILED DESCRIPTION

The present invention relates to an electroencephalographic (EEG) data analysis system and, more specifically, to a mobile in vivo brain scan system that is configured to collect remote and mobile EEG data for real-time analysis. The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the present invention is not intended to be limited to the embodiments presented, but is to be accorded the widest scope consistent with the principles and novel features disclosed herein.

In the following detailed description, numerous specific details are set forth in order to provide a more thorough understanding of the present invention. However, it will be apparent to one skilled in the art that the present invention may be practiced without necessarily being limited to these specific details. In other instances, well-known structures and devices are shown in block diagram form, rather than in detail, in order to avoid obscuring the present invention.

The reader's attention is directed to all papers and documents which are filed concurrently with this specification and which are open to public inspection with this specification, and the contents of all such papers and documents are incorporated herein by reference. All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of” or “act of” in the claims herein is not intended to invoke the provisions of 35 U.S.C. 112, Paragraph 6.

### (1) Introduction

As noted above, the present invention relates to mobile in vivo brain scan and analysis system. In operation, the present invention requires a mobile data collection subsystem and a data analysis subsystem. For clarity, the data collection subsystem will be described first, with the data analysis subsystem thereafter described.

### (2) Data Collection Subsystem

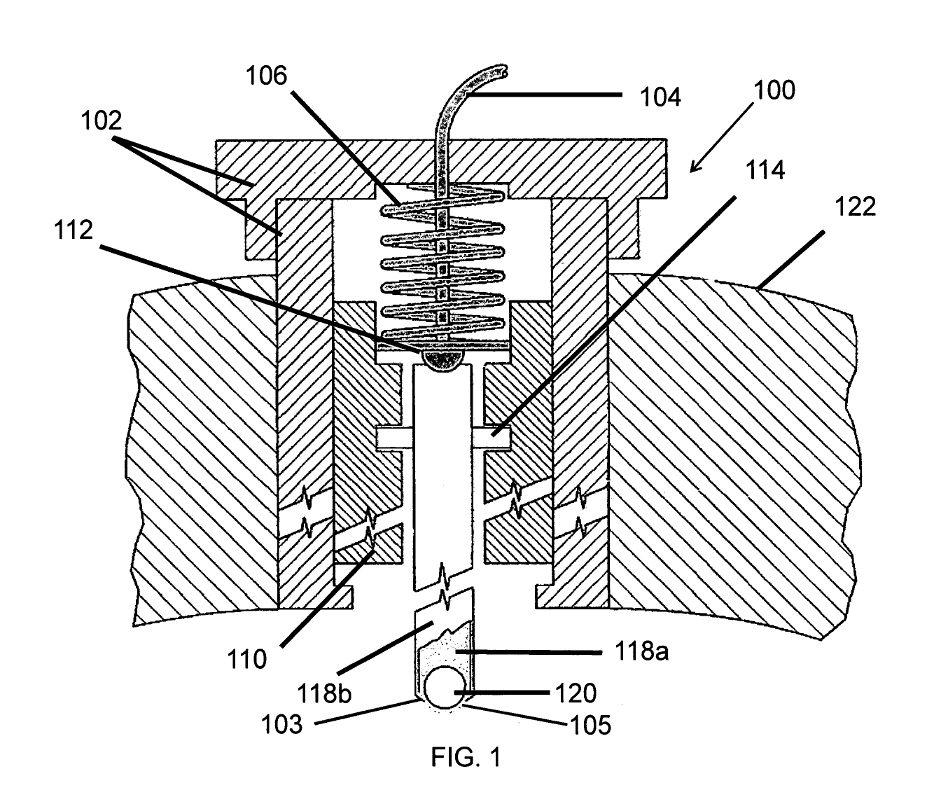

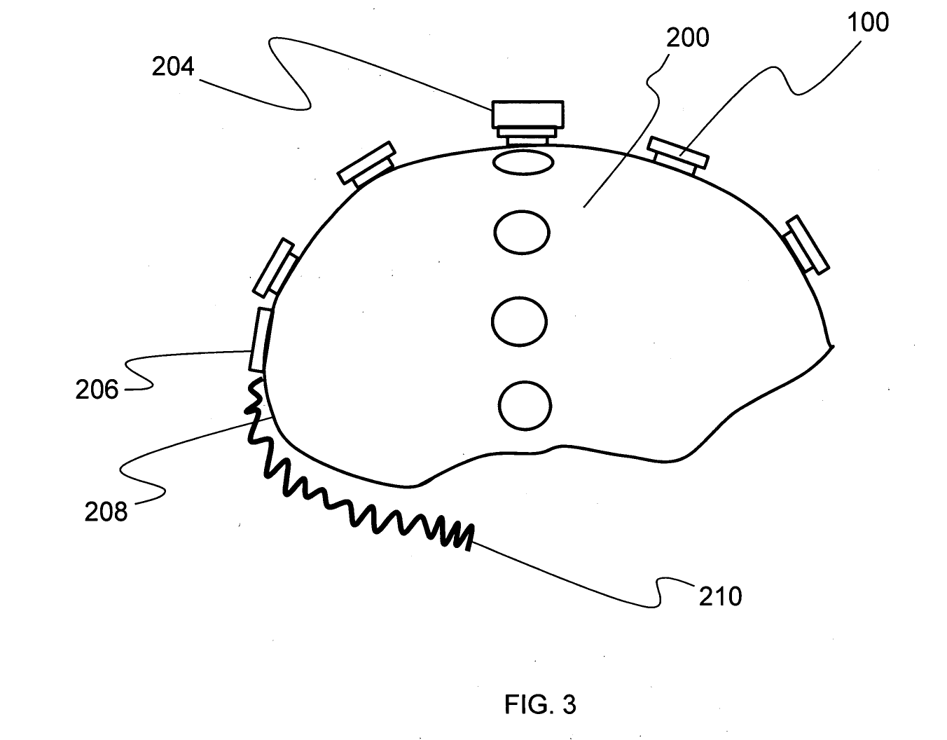

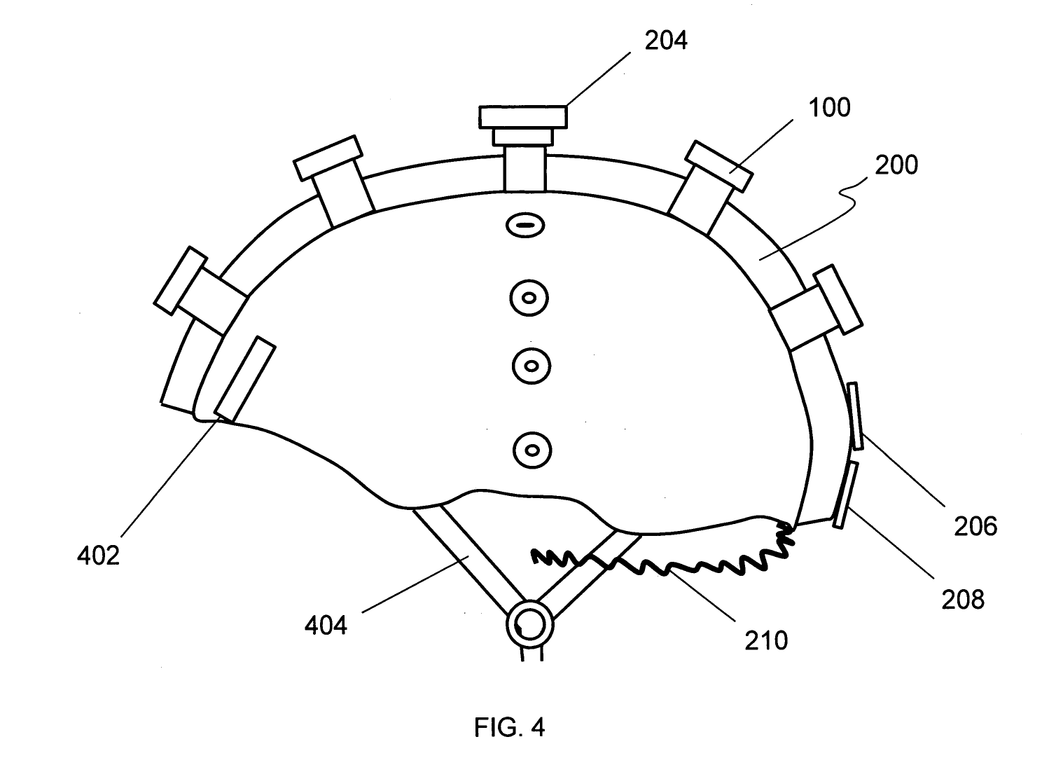

As described above, the present invention utilizes a data collection subsystem that is formed to collect brain data (e.g., EEG data) in real-time and transmit that data to a remote site for further analysis. As can be appreciated by one skilled in the art, the data collection subsystem is any suitable device that is operable for gathering brain data from a mobile subject and transmitting that data to a remote site. Thus, the data collection subsystem illustrated in FIGS. 1 through 5 provides a non-limiting example of a suitable device for operation with the present invention.

As shown in FIG. 1, the data collection subsystem includes a data collection electrode 100. For example, the data collection electrode 100 is an EEG sensor module that includes a housing 102 and a pre-loaded, conductive probe 118b attached with a slider/sleeve 110 within the housing 102. The probe 118b includes an electrically conductive base 103 with a gold or other electrically conductive roller ball (described below as the ball contact 120) for electrical communication with the scalp of a user to detect EEG signals. The probe 118b is pre-loaded (e.g., spring-loaded) such that the electrically conductive probe 118b is forced toward the scalp from the housing 102. Additionally, the probe 11b is further formed to hold a discrete amount of an electrically conductive gel 118a therein and dispense the gel 118a proximate to the electrically conductive base 103 and ball contact 120 to facilitate an electrical communication between the user's scalp and the electrically conductive base 103.

The probe 118b is also formed to have a reservoir therein for containing the electrically conductive gel 118a. In order to dispense the gel 118a, a dispensing hole 105 is formed at the electrically conductive base 103 that allows for fluidic communication from the reservoir to a user's scalp. An electrically conductive captive ball contact 120 is included within the probe 118b and extends from the hole 105. The ball contact 120 includes a diameter that is greater than a diameter of the hole 105, but less than an interior diameter of the probe 118b, thereby maintaining the ball contact 120 within the probe 118b. The hole 105 in conjunction with the ball contact 120 allows limited application of the electrically conductive gel 118a to the point of contact (i.e., the ball contact 120 and/or the base 103) of the conductive probe 118b with the user's scalp. The ball contact 120 is free to rotate and limits the flow of electrically conductive gel out of the dispensing hole 103, depending on the movement of the probe 118b. The ball contact 120 is formed of any suitably conductive material, a non-limiting example of which includes gold.

In a desirable aspect, the conductive probe 118b will be replaceable in order to easily replenish the reservoir of electrically conductive gel 118a. For example, the probe 118b is formed with a cantilevered key lock 114 to hold the probe 118b in place and to provide for easy removal and replacement. Alternatively, the conductive probe 118b can be removed to allow a user to refill the reservoir. In yet another aspect, the probe 118b is disposable, allowing for placement of a new replacement probe 118b.

To allow the probe 118b to slide within the housing 102, a slider/sleeve 110 is provided to mount the probe 118b within the housing 102. Both the slider/sleeve 110 and the outer electrode housing 102 are made with electrically insulating materials. Additionally, the slider/sleeve 110 and the housing 102 are made of materials that have a low coefficient of friction with one another to allow the slider/sleeve 110 to slide easily within the housing 102. As a non-limiting example, the slider/sleeve 110 and the housing 102 can be formed of a non-conducting material, such a Teflon™.

A contact surface 112 is attachable (using a device such as the cantilevered key lock 114 integral with the probe 118b) with the probe 118b to transmit signals from the probe 118b to a signal wire 104. The contact surface 112 is formed in any suitable shape to facilitate an electrical connection between the probe 118b and the signal wire 104. For example, the contact surface is a hemispherical electrical contact surface with displaceable shoulder that is in direct electrical contact at its proximal face with the conductive probe 118b and at its distal face with the signal wire 104.

As mentioned above, the probe 118b is slightly pre-loaded to force the probe 118b toward a user's scalp. The probe 118b is pre-loaded using any suitable mechanism or device, a non-limiting example of which includes a light spring 106. The spring 106 is in contact with the slider 110 to move the conductive probe 118b toward the user's scalp and maintain constant pressure of the electrical contact surfaces 103 and 120 of the conductive probe 118b and the electrically conductive gel to the scalp.

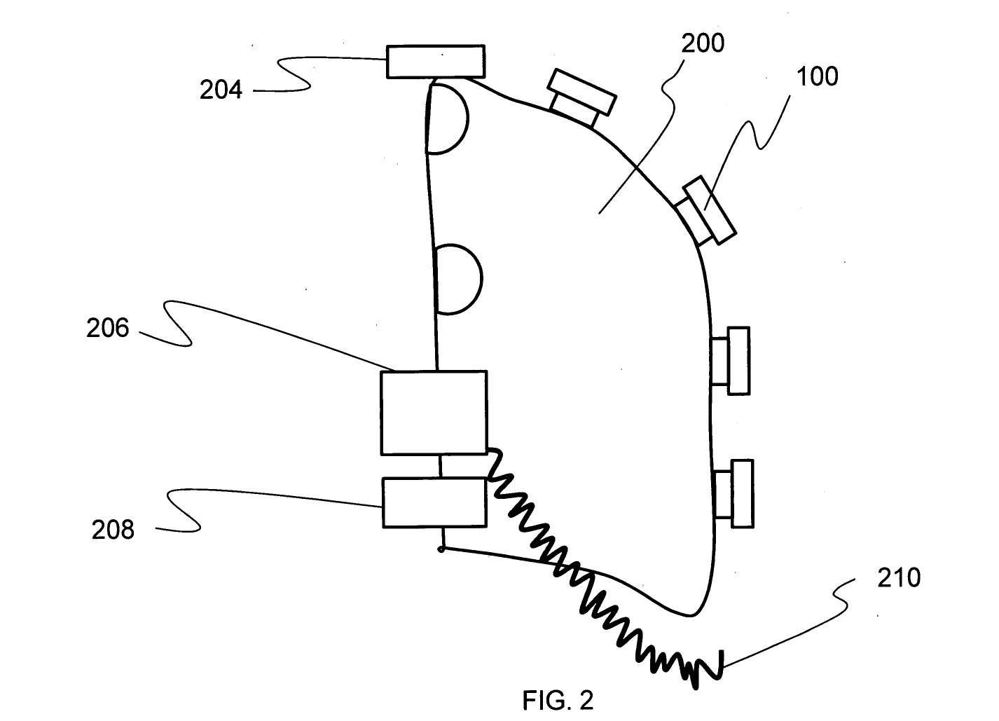

As can be appreciated by one skilled in the art, a sole electrode, in of itself, does not enable a user to capture useful EEG data. Thus, the present invention also includes a helmet with a shock absorbent lining for attaching to a user's scalp. FIGS. 2 through 4 depict various views of a helmet's shock lining 200 according to the present invention. The shock lining 200 is any suitable material that allows a user to affix a plurality of electrodes 100 to the user's scalp, a non-limiting example of which includes a standard bicycle helmet. To facilitate in vivo usage, the shock lining 200 includes shock-absorbing pads 402 and a chin-strap 404 to stabilize the shock lining 200, which stabilizes the EEG sensor assemblies (i.e., data collection electrodes).

As described in further detail below, the present invention also allows the system to transmit the multiple channel EEG data to a remote location, such as a Receiver Data Processor System, a non-limiting example of which includes a Relational Database Management System (RDBMS). To enable such a transmission, a plurality of signal wires (shown as element 104 in FIG. 1) transfer the data from the individual electrodes 100 to a Signal Processor and Transmitter 206, as shown in FIG. 2. Data will be transferred from the transmitter by use of any suitable transmission device, such as a patch antenna 204 mounted on the outside of the helmet. Additionally, the mobile EEG system (helmet shock lining 200, multiple electrodes 100, and requisite components) will be powered by a rechargeable or replaceable battery 208 or any other suitable power source. An EEG common ground lead 210 will be required which will serve as a reference for all recorded EEG data.

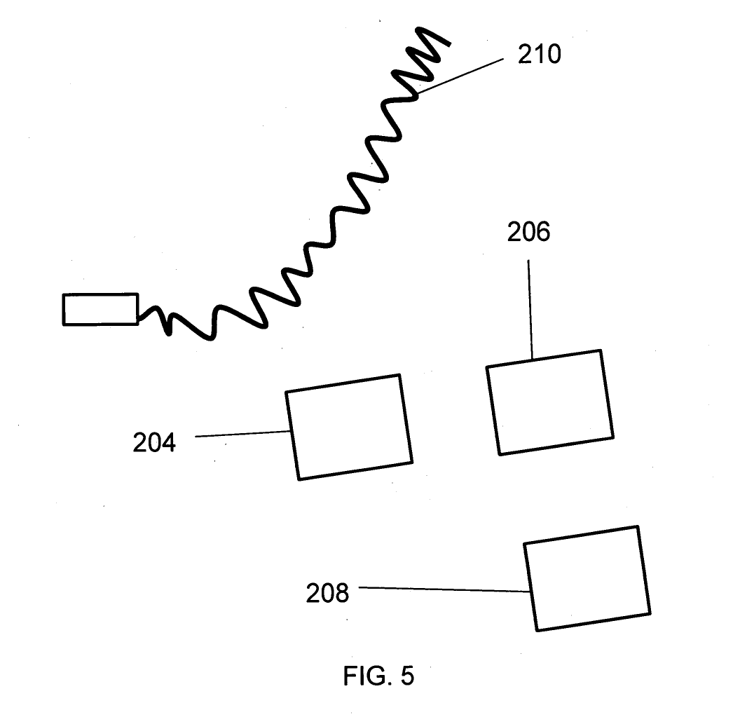

FIG. 5 further illustrates some of the important electronics utilized in the system, including the analog-to-digital Signal Processor and multiple channel Transmitter 206, the patch antenna 204 for placement outside the helmet, the battery 208, and the EEG common ground lead 210.

As a further description, the spring-loaded, ball-point-pen-like electrically conductive probe 118b is assembled into a small cylinder (i.e., housing 102) and is mounted in the shock-absorber lining of a helmet (described in further detail below). One or more of these cylinders will be used in the system.

In a desired aspect, these cylinders (e.g., typically 1.5 centimeters (cm)×1.5 cm in diameter) are mounted in such a way and in such numbers as to effectively replicate the typical placement and distribution of the standard, paste-on EEG probes used in medical and clinically based settings (or in current ambulatory EEG systems). The evoked electromotive force (EMF) wave potentials, generated from firing neuronal bundles in the brain, are picked up by these “floating” sensor probes and carried by small, insulated cables to a miniaturized multi-channel processor and radio-frequency (RF) transmitter inside the helmet and connected to typical Patch Antennas affixed to the outside surface of the helmet. Signal sampling rates can be on the order of microseconds so as to detect the multiple locations of sequentially firing neurons. These transmitted signals are received at a remote site for further processing into three-dimensional images, depicting the location of the firing neuronal bundles in three-dimensional (3 D) space, and are superimposed on a graphically depicted translucent brain model matching the size of the subject under study. The processed signals and images are then downloaded to the relational database management system (RDBMS) for further study, analysis, and comparison with other similar data.

In another aspect, the EEG (EMF) data collected by each of the data collection electrodes 100 will be passed through the small wire bundle to the helmet Signal Processor and Transmitter. The collected data is then transmitted by the small RF Transmitter to a remote location where it is downloaded into a computerized data base for further inspection, normalization, and preparation for comparison to similar data in International Brain Data Base Systems.

### (3) Data Analysis Subsystem

As noted above, the present invention also includes a data analysis subsystem that is configured to receive the brain data at a remote site for further processing. For example, the system is configured to display the data in a visual, 3 D format that will enable diagnostic professionals to identify the precise neural-physiological sources and transmission patterns of the firing neurons in real-time, which will greatly improve the understanding of the actual function of the brain as relates to actual physical and psychological behavior. It should be noted that although the present invention is described as being used with EEG data, it is not intended to be limited thereto as the data collection and analysis aspects of the present invention can be used with any measurable data that is representative of brain function.

As described above, the present invention relates to a system for mobile electroencephalographic (EEG) data collection, analysis, and 3 D display of firing neurons in the brain, otherwise known as evoked potentials. The system utilizes electrodes (an example of which is described above) that are capable of the automatic collection of EEG data. The present invention is also capable of collecting and analyzing the acquired data. In this aspect, neural activity, in the form of evoked field potentials and electromotive force (EMF) signals, will be recorded simultaneously from multiple channels. The data is transmitted to a remote site for further analysis of the raw data (as is usually done by a neurologist) and then processed by time-domain software into a 3 D display of the location of firing neurons. The acquired data will be become part of a RDBMS for EEG data and will be professionally analyzed on a time-scale that approaches real-time or near-real-time.

The present invention includes an automated diagnosis system as part of the RDBMS. Thus, the present invention includes a data analysis system that provides a means for a peer-review approach to the analysis and comparison of EEG data with other (e.g., international) RDBMS systems which contain similar data. The analysis and comparison of these brain-wave patterns and corresponding images can be made available for study by trained medical professionals or compared to other, similar signals and images and associated diagnoses located in RDBMS's at similar international research locations.

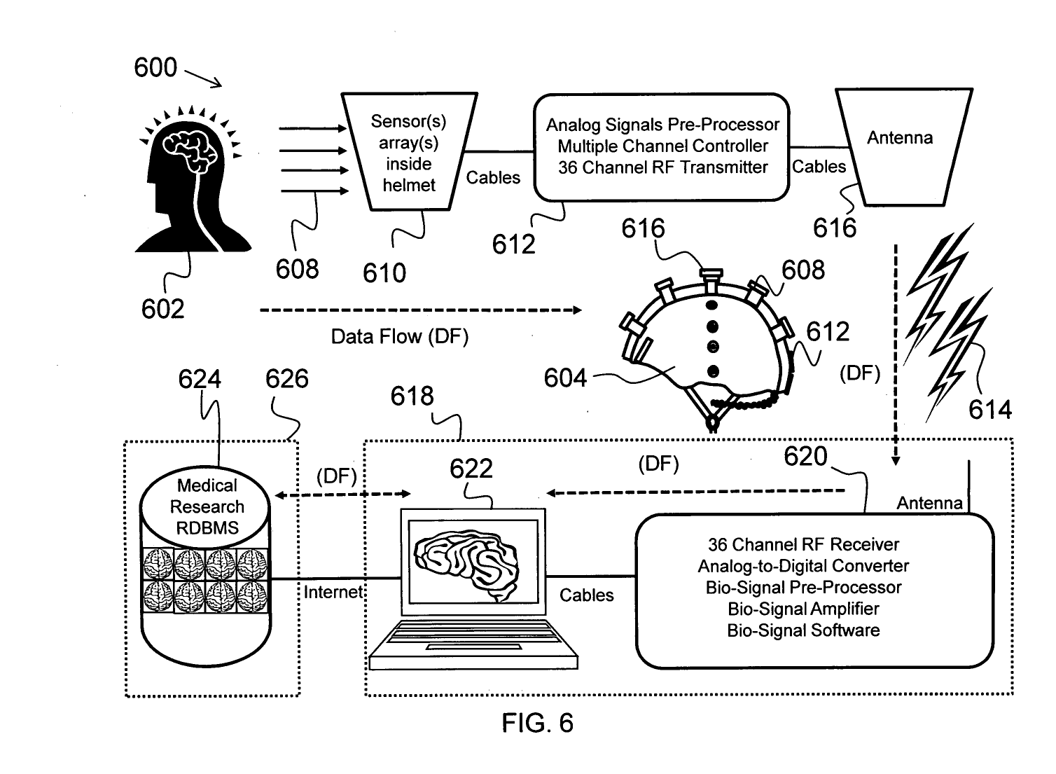

FIG. 6 illustrates the components and data flow of the mobile in vivo brain scan and analysis system 600 according to the present invention.

As shown, a mobile subject 602 under study is provided with a helmet 604 (or other suitable device) containing a plurality of data collection electrodes 606 (e.g. EEG sensors). Neuron bundles in the mobile subject's 602 brain generate evoked potentials 608 which are captured by an array 610 of data collection electrodes 606 positioned within the helmet 604. The evoked potentials 608 are then passed to a signal processor and transmitter 612. The signal processor includes a digital-to-analog pre-processor and a multi-channel controller. The transmitter is any suitable mechanism or device for transmitting said signals, a non-limiting example of which includes a 36 channel radio frequency (RF) transmitter. Thus, the signal processor and transmitter 612 converts the digital signals into analog signals 614 and further transmits the signals 614 using an antenna 616 (e.g., a patch antenna attached on the outside of the helmet). The helmet 604, sensor array 610 (including electrodes 606), signal processor and transmitter 612, and antenna 616 collectively operate as a non-limiting example of a data collection system according to the present invention.

The signals 614 are then transmitted to the data analysis system. The signals 614 are captured at a remote site 618 using a receiver system 620. The receiver system 620 is any suitable mechanism or device capable of initially receiving the signals 614 and pre-processing the signals 614 for further processing. As a non-limiting example, the receiver system 620 includes a 36 channel RF receiver, an analog-to-digital converter, a bio-signal pre-processor, a bio-signal amplifier, and bio-signal software. The 36 channel RF receiver is used to receive the various signals 614 as transmitted by the RF transmitter. The analog-to-digital converter is used to convert the captured analog signals into digital signals.

The bio-signal pre-processor is used to prepare the signals to convert the analog EMF signals coming from the evoked potentials in the brain to digital signals used to transmit the data to the remote site. The bio-signal amplifier is used to amplify the signals for further processing. The bio-signal software is used for taking the 36 channels of data.

The receiver system 620 is connected with a data processing system 622 (e.g., computer with one or more processors) for further processing. The receiver system 620 can be incorporated into the same machine as the data processing system 622. The data processing system 622 includes 3 D software and RDBMS controller for connecting with and operating an RDBMS 624. The RDBMS 624 is connected with the data processing system 622 through any suitable communicative connection, non-limiting examples of which include being directly hard-wired, being connected through the Internet, and a wireless connection. As discussed above, a desired aspect of the present invention is that it provides for the collection of data and access to that data remotely. Thus, it is desirable that the data processing system 622 is connected with the RDBMS 624 through the Internet, with the RDBMS 624 being at yet another remote site 626 (although in another aspect, the RDBMS 624 can be directly connected to the data processing system 622).

Additionally, it should be noted that although cables are listed as the connection device between several of the components illustrated in FIG. 6, the invention is not intended to be limited thereto as any other suitable communicative connection can be established between the various illustrated components, non-limiting examples of which include wireless and Bluetooth connections.

These signals are processed for transmission by a small integrated multi-channel transmitter to a remote site for further computer processing into three-dimensional (3 D) images which show the location (with millimeter accuracy) and the sequential timing (in microseconds) of these firing neurons. The frequency and power of the small, helmet-integrated transmitter is designed within the narrow range of non-bio-harmful parameters. The 3 D images are produced using any suitable signal interferometric technique. A non-limiting example of such a technique is the postulated Boundary Element Method, such as that described by Stefan F. Filipowicz in “Identification of the Internal Sources with the Aid of Boundary Element Method,” as published at the International Workshop entitled, “Computational Problems of Electrical Engineering,” Zakopane, Poland, 2004, which is incorporated by reference as though fully set forth herein.

Thus, the brain data (EEG data) can be used to construct 3 D images of evoked potentials within the brain. As a non-limiting example, initially assuming homogeneity of the transport and diffusion mechanisms of the cellular structures under study, the governing equations are (1) Poisson's equation with (2) Neumann's boundary conditions, according to the following:

$$\begin{matrix}{∇}^{2} u ( \vec{r} ) = - b ( \vec{r} ) , \\ and & ( 1 ) \\ q ( \vec{r} ) \frac{δ u ( \vec{r} )}{δ n} = 0 , & ( 2 )\end{matrix}$$

where u denotes electric potential, b denotes internal sources, and denotes a position vector.

The discrete, individual, evoked potential data collected by the data collection subsystem can be presented in a 3 D format using a postulated Boundary Element Method or some other method, such as the least squares method. As such, the present invention is configured to use the Boundary Element Method to mathematically model and depict the firing neuronal bundles in the brain, in 3 D space, integrated with a semi-translucent model of the brain. In other words, the brain data is displayed in a 3 D form, integrated with a semi-transparent or semi-translucent model of the brain as it approximates the subject's actual brain size, thereby improving 3 D visualization.

The 3 D images produced are comparable to and look similar to functional magnetic resonance imaging (fMRI), but in an active environment and in real-time. The data is collected while the subject is mobile and functioning in a normal work or play environment. This is to be contrasted with fMRI data collection techniques which require several minutes to obtain sufficient data for display. Thus, the subjects under study (in fMRI) must remain immobilized during the entire procedure. Alternatively, the present invention uses a remote, mobile, in vivo data collection subsystem which, in combination with the data analysis subsystem, can generate and display the relevant images in milliseconds, which is more consonant with the firing rate of neurons in the Central Nervous System (CNS).

The processed images are capable of inter-active, three-dimensional manipulation and examination. The processed data can be viewed in real time and also be compared with a library of brain data (such as a relational data base management system (RDBMS)), through the Internet, to similar data existing in international medical and research databases, such as the Laboratory on Neural Imaging (LONI) at the University of California, Los Angeles (UCLA), for comparison and validation of brain function diagnoses.

As can be appreciated by one skilled in the art, the present invention covers a wide range of brain imaging applications; such as medical triage events, physical, psychological, or other trauma.

The local and remotely controlled RDBMS 624 will allow for professional cooperative collaboration in the diagnosis of abnormal neural functioning that is indicative of pathology. For example, the brain data can be compared with a library of brain data to identify any anomalies in the brain data that may be indicative of a particular malady or pathology (abnormal brain function). Should such an anomaly be identified, it is possible to compare the anomaly with a database to diagnose the anomaly and notify the user of such an anomaly and/or diagnosis.

It is a goal of the present invention to create a system for both local-immediate and automated classification of, or hypothesis generation for, possible diagnosis of subjects under study 602. The automatic classification of acquired data having traits that are consistent with certain pathologies can be achieved by directly generating (through software) a classification using markers that are decided upon via a professional collaborative effort. An alternative is to build a system that employs some form of artificial intelligence or machine learning to perform the classification. Support Vector Machines, Bayesian Networks, and in general Knowledge Based Systems are examples of possible methods that allow a system to classify acquired data as being indicative of some pathology without the need to discreetly describe all of the classification rules.

In summary, the present invention comprises a new EEG data collection system that allows for mobile, in vivo EEG data collection, analysis, and diagnosis of EMF brain patterns. To accomplish this, evoked potentials (EEG), generated by firing neuronal bundles in the brain, are detected by the sensors (i.e., the data collection electrodes), gently riding on the surface of the scalp. These signals are processed for transmission by a small integrated multi-channel transmitter to a remote site for further computer processing into three-dimensional (3 D) images which show the location (with millimeter accuracy) and the sequential timing (in microseconds) of these firing neurons. The processed images are capable of inter-active, three-dimensional manipulation and examination. The processed data can be viewed in real time and also be compared via a relational data base management system (RDBMS), through the Internet, to similar data existing in international medical and research databases for analysis and diagnosis.

What is claimed is:

1. A mobile brain scan and analysis system, comprising:
a data analysis subsystem, the data analysis subsystem including one or more processors that are configured to receive brain data from a remote data collection subsystem, the brain data being reflective of firing neurons in a mobile subject, and wherein the data analysis subsystem is further configured to generate and display a three-dimensional image that depicts a location of the firing neurons.

2. A mobile brain scan and analysis system as set forth in claim 1, wherein the data analysis subsystem is further configured to:
compare the brain data against a library of brain data to detect an anomaly in the brain data, the anomaly being indicative of an abnormal brain function; and
notify a user of any detected anomaly in the brain data.

3. A mobile brain scan and analysis system as set forth in claim 2, further comprising a data collection subsystem, the data collection subsystem being formed to collect brain data and transmit the brain data to the data analysis subsystem.

4. A mobile brain scan and analysis system as set forth in claim 3, wherein the data collection subsystem is further formed to transmit the brain data wirelessly to the data analysis subsystem.

5. A mobile brain scan and analysis system as set forth in claim 4, wherein data collection subsystem further comprises a helmet with an array of electrodes, with each of the electrodes being formed to detect electroencephalograph (EEG) data from a user.

6. A mobile brain scan and analysis system as set forth in claim 5, wherein the data analysis subsystem further comprises:
a receiver system, the receiver system being configured to receive the transmitted brain data from the data collection subsystem;
a data processing system, the data processing system having a relational database management system (RDBMS) controller for connecting with and operating an RDBMS having a library of brain data, and further being configured to receive the brain data from the receiver system and compare the brain data to the RDBMS to detect an anomaly in the brain data.

7. A mobile brain scan and analysis system as set forth in claim 6, wherein the data analysis subsystem is further configured to compare a detected anomaly in the brain data with an RDBMS to generate a diagnosis of the detected anomaly.

8. A mobile brain scan and analysis system as set forth in claim 7, wherein the data analysis subsystem is further configured to compare the three-dimensional image with a RDBMS having a library of three-dimensional images to detect an anomaly in the brain data.

9. A mobile brain scan and analysis system as set forth in claim 1, further comprising a data collection subsystem, the data collection subsystem being formed to collect brain data and transmit the brain data to the data analysis subsystem.

10. A mobile brain scan and analysis system as set forth in claim 9, wherein the data collection subsystem is further formed to transmit the brain data wirelessly to the data analysis subsystem.

11. A mobile brain scan and analysis system as set forth in claim 9, wherein data collection subsystem further comprises a helmet with an array of electrodes, with each of the electrodes being formed to detect electroencephalograph (EEG) data from a user.

12. A mobile brain scan and analysis system as set forth in claim 9, wherein the data analysis subsystem further comprises:
a receiver system, the receiver system being configured to receive the transmitted brain data from the data collection subsystem;
a data processing system, the data processing system having a relational database management system (RDBMS) controller for connecting with and operating an RDBMS having a library of brain data, and further being configured to receive the brain data from the receiver system and compare the brain data to the RDBMS to detect an anomaly in the brain data.

13. A mobile brain scan and analysis system as set forth in claim 1, wherein the data analysis subsystem is further configured to compare a detected anomaly in the brain data with a relational database management system (RDBMS) to generate a diagnosis of the detected anomaly.

14. A mobile brain scan and analysis system as set forth in claim 1, wherein the data analysis subsystem is further configured to compare the three-dimensional image with a relational database management system (RDBMS) having a library of three-dimensional images to detect an anomaly in the brain data.

15. A method for detecting anomalous brain activity, comprising acts of:
receiving brain data from a remote data collection subsystem, the brain data being reflective of firing neurons in a mobile subject; and
generating and displaying a three-dimensional image with a data analysis subsystem that depicts a location of the firing neurons.

16. A method as set forth in claim 15, further comprising acts of:
comparing the brain data against a library of brain data to detect an anomaly in the brain data, the anomaly being indicative of an abnormal brain function; and
notifying a user of any detected anomaly in the brain data.

17. A method as set forth in claim 15, further comprising acts of:
collecting the brain data with the remote data collection subsystem; and
transmitting the brain data wirelessly to the data analysis subsystem.

18. A method as set forth in claim 15, further comprising an act collecting the brain data using a helmet with an array of electrodes, with each of the electrodes being formed to detect electroencephalograph (EEG) data from a user.

19. A method as set forth in claim 15, further comprising acts of:
receiving the transmitted brain data from the data collection subsystem; and
comparing the brain data to a relational database management system (RDBMS) to detect an anomaly in the brain data; and
generating a diagnosis of the detected anomaly.

20. A method as set forth in claim 15, further comprising an act of:
comparing the three-dimensional image with a relational database management system (RDBMS) having a library of three-dimensional images to detect an anomaly in the brain data.
